(19) United States
(12) Patent Application Publication
(10) Pub. No.: US 2022/0062034 A1
(43) Pub. Date: Mar. 3, 2022

(54) LASER SURGICAL SYSTEMS AND METHODS FOR CREATING A MARKER IN AN EYE

(71) Applicant: Alcon Inc., Fribourg (CH)

(72) Inventor: Mario Abraham, Burgthann (DE)

(21) Appl. No.: 17/393,066

(22) Filed: Aug. 3, 2021

Related U.S. Application Data
(60) Provisional application No. 63/070,775, filed on Aug. 26, 2020.

(51) Int. Cl. A61F 9/008; A61B 90/00; G16H 20/40; G08B 7/06

(52) U.S. Cl. CPC A61F 9/008; A61B 90/361; A61F 9/00836; A61F 9/00827; A61B 2017/00119; G08B 7/06; A61F 2009/00872; A61F 2009/00897; G16H 20/40

(57) ABSTRACT

In certain embodiments, an ophthalmic surgical system for creating a marker in a cornea includes controllable components, a camera, and a computer. The controllable components include a laser source, a scanner, and an objective. The laser source generates a laser beam. The scanner transversely and longitudinally directs a focal point of the laser beam. The objective focuses the focal point towards the eye. The camera images movement of the eye. The computer creates the marker by: instructing the scanner to direct the focal point towards a peripheral region of the cornea; and instructing the controllable components to create the marker in the peripheral region. The computer also determines that movement of the marker is in an alert range indicating an unacceptable amount of movement, and provides notifications in response to determining that the movement of the marker is in the alert range.

### TECHNICAL FIELD

The present disclosure relates generally to laser surgical systems and methods, and more particularly to laser surgical systems and methods for creating a marker in an eye.

### BACKGROUND

Certain ophthalmic laser surgical systems generate a pulsed laser beam to perform a surgical procedure on an eye. In some procedures, the laser beam creates photodisruptions at specific points in the eye according to a treatment pattern. The laser beam should be properly aligned with the eye throughout the procedure to create photodisruptions that precisely match the pattern.

A patient interface (PI) is usually used to hold the eye in position during the procedure. The patient interface is typically affixed to the eye by a vacuum to secure the eye in place to facilitate proper alignment of the eye with the treatment pattern during the procedure.

### BRIEF SUMMARY

In certain embodiments, an ophthalmic surgical system for creating a marker in a cornea for a surgical procedure includes controllable components, a camera, and a computer. The controllable components include a laser source, a scanner, and an objective. The laser source generates a laser beam having ultrashort pulses. The scanner transversely and longitudinally directs a focal point of the laser beam. The objective focuses the focal point through a patient interface towards the eye. The camera images movement of the eye. The marker has a shape indicating rotational movement of the eye. The computer creates the marker by: instructing the scanner to transversely and longitudinally direct the focal point towards a peripheral region of the cornea; and instructing one or more of the controllable components to create the marker in the peripheral region of the cornea. The computer also determines that movement of the marker is in an alert range indicating an unacceptable amount of movement, and provides one or more notifications in response to determining that the movement of the marker is in the alert range.

Embodiments may include none, one, some, or all of the following features:

*The shape of the marker is selected from one or more of the following: a polygon, a line, a plurality of lines, a plurality of lines intersecting at a point, a plurality of lines intersecting at a plurality of points, a circle with a line, an oval, and one or more alphanumeric characters.

*The computer identifies a marker corresponding to the surgical procedure and creates the identified marker in the cornea. In certain embodiments, if the surgical procedure is a lenticule extraction procedure, the computer identifies that the marker is external to and proximate to an outer boundary of a lenticule to be extracted, and creates the marker external to and proximate to the outer boundary. In certain embodiments, if the surgical procedure is a flap creation procedure, the computer identifies that the marker is external to and proximate to an outer boundary of a flap to be created, and creates the marker external to and proximate to the outer boundary. In certain embodiments, if the surgical procedure is a cataract removal procedure, the computer identifies that the marker outlines a lens capsule of the eye, and creates the marker that outlines the lens capsule of the eye.

*A notification may be an audio notification.

*A notification may be a visual notification.

*The alert range comprises a plurality of non-overlapping subset alert ranges that form a partition of the alert range. The computer provides a plurality of notifications. Each notification corresponds to a subset alert range, where a first notification for a first subset alert range is distinct from a second notification for a second subset alert range. In certain embodiments, the first notification has a first visual characteristic that is distinct from a second visual characteristic of the second notification. In certain embodiments, the first notification has a first audio characteristic that is distinct from a second audio characteristic of the second notification. In certain embodiments, a subset alert range is a terminating alert range, and the computer terminates the surgical procedure in response to determining that the movement of the marker is in the terminating alert range. In certain embodiments, the ophthalmic surgical system includes a display screen. The computer displays a notification icon comprising alert levels on the display screen, where an alert level corresponds to a subset alert range, and visually emphasizes an alert level in response to determining that the movement of the marker is in the corresponding subset alert range.

*The computer determines the alert range corresponding to the surgical procedure. In certain embodiments, if the surgical procedure is a lenticule extraction procedure, the alert range has a maximum acceptable distance of 50 to 150 microns. In certain embodiments, if the surgical procedure is a flap creation procedure, the alert range has a maximum acceptable distance of 100 to 500 microns. In certain embodiments, if the surgical procedure is a cataract removal procedure, the alert range has a maximum acceptable distance of 100 to 500 microns.

In certain embodiments, a method for creating a marker in a cornea of an eye for a surgical procedure includes creating, by a computer, the marker in the cornea. The marker has a shape that can indicate rotational movement of the eye. The computer instructs a scanner to transversely and longitudinally direct a focal point of a laser beam towards a peripheral region of the cornea, and instructs one or more of a set of controllable components to create the marker in the peripheral region of the cornea. The set of controllable components comprises a laser source configured to generate the laser beam, the scanner configured to transversely and longitudinally direct the focal point of the laser beam, and an objective configured to focus the focal point through a patient interface towards the eye. The computer also determines that movement of the marker is in an alert range, the alert range indicating an unacceptable amount of movement of the marker, and provides one or more notifications in response to determining that the movement of the marker is in the alert range.

Embodiments may include none, one, some, or all of the features described above with respect to the system and/or none, one, some, or all of the following features:

*The method further includes identifying the marker corresponding to the surgical procedure, and creating the identified marker in the cornea.

*The alert range comprises a plurality of non-overlapping subset alert ranges that form a partition of the alert range. The method further includes: providing a plurality of notifications, each notification corresponding to a subset alert range, a first notification for a first subset alert range distinct from a second notification for a second subset alert range; displaying a notification icon comprising a plurality of alert levels on a display screen, at least one alert level corresponding to a subset alert range; and visually emphasizing an alert level in response to determining that the movement of the marker is in the corresponding subset alert range.

### BRIEF DESCRIPTION OF THE DRAWINGS

FIG. 1 illustrates an example of an ophthalmic surgical system configured to create a marker in the cornea of an eye according to certain embodiments;

FIGS. 2A and 2B illustrate an example of a marker created in an eye;

FIG. 3 illustrates a marker that includes circles with a line;

FIG. 4 illustrates a marker that includes alphanumeric characters;

FIG. 5 illustrates a marker that includes lines;

FIG. 6 illustrates a marker that includes lines and a circle;

FIG. 7 illustrates an example of a traffic light notification icon that may be used by the system of FIG. 1;

FIG. 8 illustrates an example of a meter notification icon that may be used by the system of FIG. 1; and

FIG. 9 illustrates an example of a method for creating a marker in an eye that may be performed by the system of FIG. 1.

### DESCRIPTION OF EXAMPLE EMBODIMENTS

Referring now to the description and drawings, example embodiments of the disclosed apparatuses, systems, and methods are shown in detail. The description and drawings are not intended to be exhaustive or otherwise limit the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments.

In certain ophthalmic surgical procedures, the eye is coupled to a surgical system with a patient interface (PI) in order to properly align the eye with a treatment pattern. An issue may arise in that the eye might move relative to the patient interface, and thus relative to the treatment pattern. For example, a nervous patient may vigorously move their eyes, creating a shear force strong enough for the eye to move relative to the patient interface or even become disconnected from the interface. As another example, an inexperienced surgeon may inadvertently move the eye relative to the patient interface or may fail to properly connect the eye to the patient interface such that the eye moves relative to the interface. Movement of the eye relative to the patient interface may cause the eye to be misaligned with the treatment pattern. Embodiments described herein may address this issue.

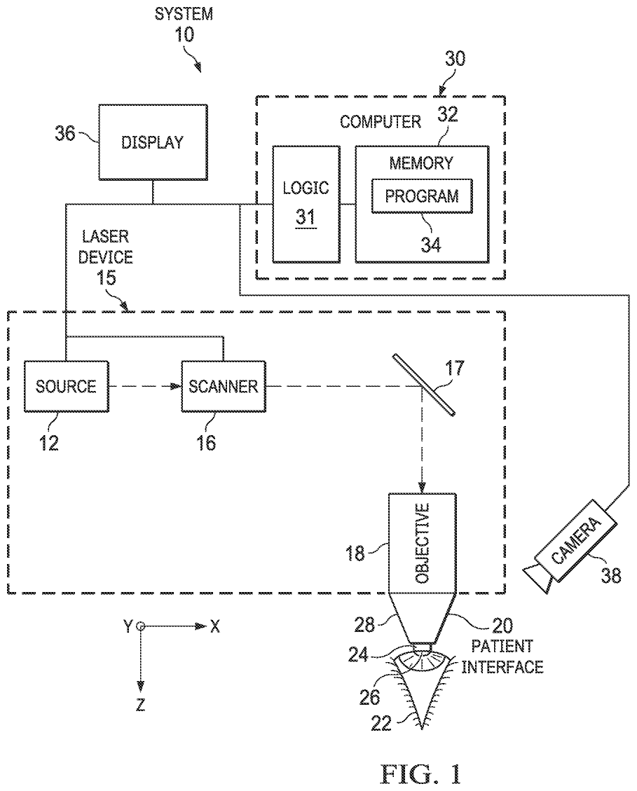

FIG. 1 illustrates an example of an ophthalmic surgical system 10 configured to create a marker in the cornea of an eye 22 according to certain embodiments. In the embodiments, system 10 creates the marker in the cornea, tracks the movement of the marker, and provides a notification if the movement is in an alert range indicating that the eye has moved relative to the patient interface. Accordingly, system 10 may be used to address the issue of eye 22 moving relative to the patient interface and becoming misaligned with the treatment pattern.

In the illustrated example, system 10 includes a laser device 15, a patient interface 20, a camera 38, and a control computer 30, coupled as shown. Laser device 15 includes controllable components, such as a laser source 12, a scanner 16, one or more optical elements 17, and/or a focusing objective 18, coupled as shown. Patient interface 20 includes a contact portion 24 (with an abutment face 26) and a sleeve 28 coupled as shown. Computer 30 includes logic 31, a memory 32 (which stores a computer program 34), and a display 36, coupled as shown.

As an overview, system 10 can create and track the marker according to the following example of operation. Laser source 12 generates a laser beam with ultrashort pulses. Scanner 16 transversely and longitudinally controls a focal point of the laser beam. Objective 18 focuses the focal point through patient interface 20 towards eye 22. Camera 38 images movement of eye 22. Computer 30 creates the marker in the cornea by instructing scanner 12 to direct the focal point towards a peripheral region of the cornea and by instructing the controllable components to create the marker in the peripheral region. Computer 30 then determines whether the movement of the marker is in an alert range describing unacceptable movement of the marker. If so, computer 30 provides one or more notifications that the movement is in the alert range.

Turning to the parts of system 10, laser source 12 generates a laser beam with ultrashort pulses. An ultrashort pulse refers to a light pulse that has a duration that is less than a nanosecond, such as on the order of picoseconds, femtoseconds, or attoseconds. The laser beam may have any suitable wavelength, such as a wavelength in the range of 300 to 1500 nanometers (nm), e.g., a wavelength in the range of 300 to 650, 650 to 1050, 1050 to 1250, and/or 1250 to 1500 nm, such as 340 to 350 nm, e.g., 347 nm±1 nm. The focal point of the laser beam may create a laser-induced optical breakdown (LIOB) in tissue (e.g., the cornea) to yield a photodisruption in the tissue. The laser beam may be precisely focused to yield precise photodisruptions, which may reduce or avoid unnecessary destruction of other tissue.

Scanner 16 transversely and longitudinally directs the focal point of the laser beam. The longitudinal direction refers to the direction of the laser beam propagation, also known as the z-direction. The transverse direction refers to directions orthogonal to the direction of beam propagation, also known as the xy-plane. In certain embodiments, abutment face 26 of patient interface 20 is selected as the xy-plane at z=0.

Scanner 16 may transversely direct the laser beam in any suitable manner. For example, scanner 16 may include a pair of galvanometrically-actuated scanner mirrors that can be tilted about mutually perpendicular axes. As another example, scanner 16 may include an electro-optical crystal that can electro-optically steer the laser beam. Scanner 16 may longitudinally direct the laser beam in any suitable manner. For example, scanner 16 may include a longitudinally adjustable lens, a lens of variable refractive power, or a deformable mirror that can control the z-position of the beam focus. The components of scanner 16 may be arranged in any suitable manner along the beam path, e.g., in the same or different modular units.

One (or more) optical elements 17 direct the laser beam towards focusing objective 18. An optical element 17 can act on (e.g., transmit, reflect, refract, diffract, collimate, condition, shape, focus, modulate, and/or otherwise act on) a laser beam. Examples of optical elements include a lens, prism, mirror, diffractive optical element (DOE), holographic optical element (HOE), and spatial light modulator (SLM). In the example, optical element 17 is a mirror. Focusing objective 18 focuses the focal point of laser beam through the patient interface 20 towards a point of eye 22. In the example, focusing objective 18 is an objective lens, e.g., an f-theta objective.

Patient interface 20 interfaces with the cornea of eye 22 to couple eye 22 to laser device 15. In the example, patient interface 20 has sleeve 28 coupled to contact portion 24. Sleeve 28 detachably couples to focusing objective 18. Contact portion 24 may be translucent or transparent to the laser beam and has an abutment face 26 that interfaces with the cornea. In certain embodiments, abutment face 26 is planar and forms a planar area on the cornea, which may define the xy-plane. In other embodiments, abutment face 26 need not be planar, e.g., may be convex or concave.

Camera 38 records images of the movement of eye 22, which includes movement of the marker created in eye 22. Examples of camera 38 include a video, optical coherence tomography (OCT), or eye-tracking camera. Camera 38 delivers image data, which represent recorded images of the eye 22, to computer 30. Computer 30 carries out image processing on the image data to determine movement of the marker. The image processing includes recognizing the marker in the recorded images, determining the position of the marker, and comparing the positions of the marker imaged at different times to determine the movement of the marker.

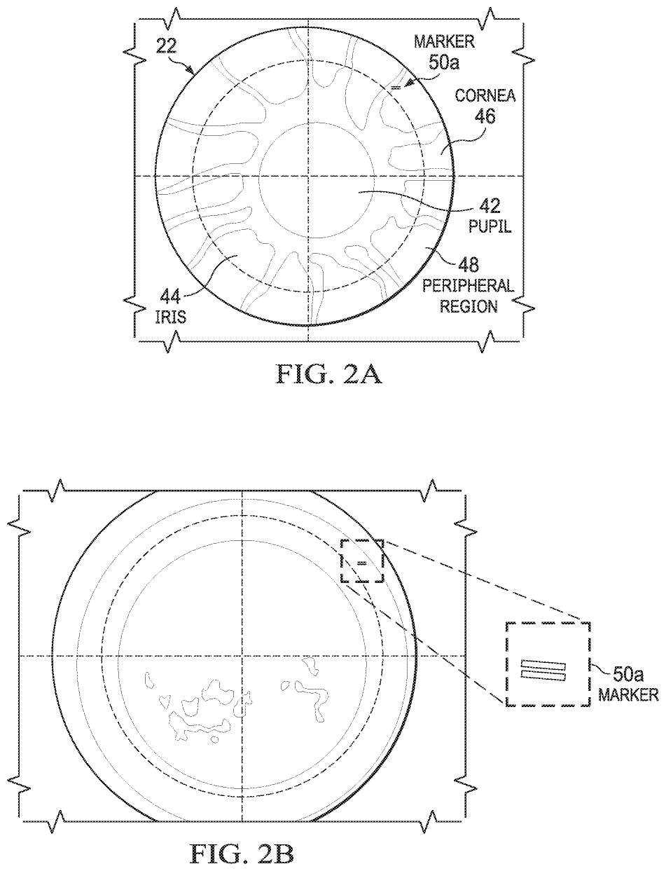

Computer 30 controls controllable components (e.g., laser source 12, scanner 16, optical elements 17, and/or focusing objective 18) in accordance with computer program 34. Computer program 34 includes computer code that instructs the controllable components to focus the laser beam at a region of the cornea and to photodisrupt at least a portion of the region to create a marker in the cornea. The marker is temporary and disappears on its own. Markers are described in more detail with reference to FIGS. 2A to 6.

FIGS. 2A and 2B illustrate an example of a marker 50 (50a) created in eye 22. FIG. 2A shows eye 22 with a pupil 42, an iris 44, a lens (not shown), and a cornea 46 with a peripheral region 48. In general, iris 44 and cornea 46 each have an outer diameter in the range of 10 to 13 millimeters (mm). Cornea 46 has a peripheral region 48, which is an annular region of cornea 46 closest to the outer perimeter of cornea 46. Peripheral region 48 may have an outer radius of Rc and an inner radius of Rc x q, where Rc represents the outer radius of cornea 46, and q is any suitable percentage, e.g., 50 to 60, 60 to 70, 70 to 80, 80 to 90, and/or 90 to 95 percent.

The inner radius may be determined in accordance with the surgical procedure. In some cases, the inner radius may be such that peripheral region 48 does not interfere with (e.g., is outside of) the treatment area of the procedure. For example, a typical lenticule diameter is approximately 5 to 7 mm, and a typical flap diameter is approximately 7 to 10 mm. The inner radius may be selected such that peripheral region 48 is outside of the lenticule or flap region. In other cases, the inner radius may be such that peripheral region 48 includes or forms a border for a surgical procedure. For example, a marker 50 may be used to outline a lens capsule, which has a diameter of approximately 9 to 10 mm. The inner radius may be selected to be less than or to match the radius of the lens capsule.

Marker 50 may have any suitable size and shape. In certain embodiments, marker 50 has a size and shape that can readily indicate translation and/or rotation of eye 22. For example, marker 50 has size sufficiently large such that camera 38 can readily detect the movement of marker 50, but sufficiently small to minimize photodisruptions of eye 22. As another example, marker 50 has a shape such that camera 38 can detect translation and/or rotation of marker 50.

Marker 50 may have any suitable location in peripheral region 48. In certain embodiments, the location of marker 50 may be determined according to the surgical procedure, such as relative to the treatment zone and/or field of view of the procedure. For example, in a lenticule extraction procedure, the treatment zone, i.e., the lenticule, may be up to approximately 8 millimeters (mm) in diameter, and the field of view may be approximately 12 mm in diameter. Marker 50 may be outside of and proximate to (e.g., within 0.010 to 2 mm, such as 0.015 mm) the outer boundary of the lenticule, but within the field of view. As another example, for a flap creation procedure, marker 50 may be outside of and proximate to the outer boundary of the flap. As yet another example, for a cataract removal procedure, marker 50 may outline the lens capsule of the eye.

Examples of shapes include: a polygon (e.g., a triangle or square), a line, a plurality of lines (e.g., an equal sign), a plurality of lines intersecting at a point (e.g., a plus sign, a cross, or an asterisk), a plurality of lines intersecting at a plurality of points (e.g., a star), a circle with a line, an oval, and one or more alphanumeric characters (e.g., text with letters of any language, numbers, and/or symbols). In the illustrated example, marker 50a is shaped like an equal sign. FIG. 2A shows marker 50a with zero rotation, such that the equal sign is horizontal. FIG. 2B shows marker 50a that has been rotated 5 degrees, such that the equal sign is tilted at an angle.

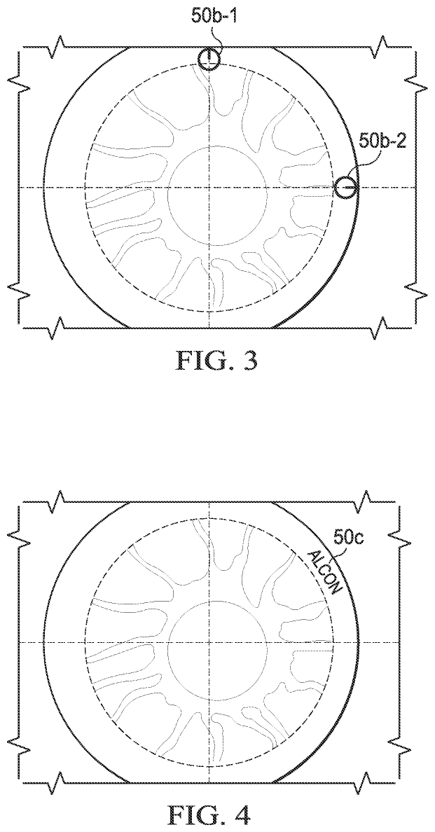

FIGS. 3 to 6 illustrate different types of markers 50. FIG. 3 illustrates a marker 50 that includes markers 50b (50b-1, 50b-2), where each marker 50b is a circle with a line. The line of marker 50b-1 is aligned with a vertical line through a center of eye 22 (e.g., pupil center, eye apex, or eye vertex), and the line of marker 50b-2 is aligned with a horizontal line through the center.

FIG. 4 illustrates a marker 50c that includes alphanumeric characters. In certain embodiments, the characters may provide information, e.g., a time stamp, a patient identifier, or a company name.

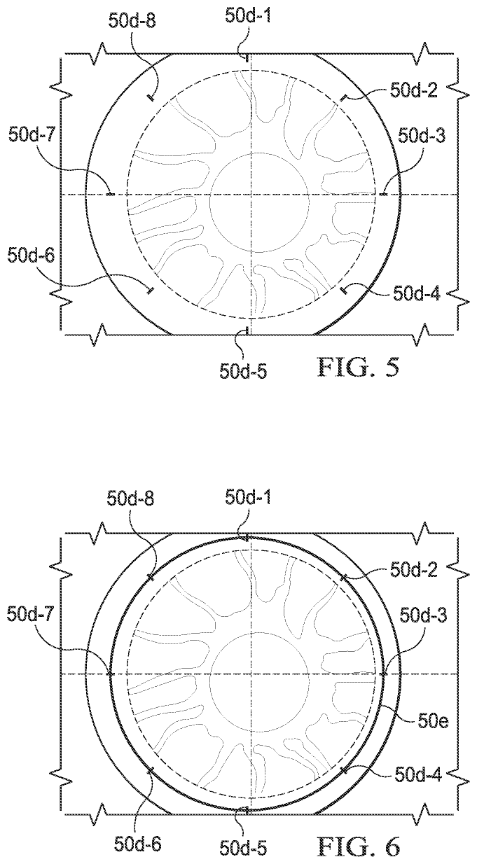

FIG. 5 illustrates a marker 50 that includes markers 50d (50d-1 to 50d-8). Each marker 50d is a line that is aligned with a line that passes through the center of eye 22. For example, markers 50d-1 and 50d-5 are aligned with a vertical line through the center, and markers 50d-3 and 50d-7 are aligned with a horizontal line through the center.

FIG. 6 illustrates a marker 50 that includes markers 50d and a marker 50e. Marker 50e is a circle that may provide a border for a treatment area, e.g., provide an outline for a lenticule, flap, or lens capsule.

Returning to FIG. 1, recall that computer 30 instructs the controllable components of laser device 15 to create marker 50. In certain embodiments, computer 30 determines the type of marker 50 to create by identifying the marker 50 corresponding to the surgical procedure. In the embodiments, particular features of a marker 50 (e.g., size, shape, location, and/or position) may be more appropriate for a specific surgical procedure. For example, computer 30 may determine that: (1) for a lenticule extraction procedure, marker 50 may be outside of and proximate to the outer boundary of the lenticule; (2) for a flap creation procedure, marker 50 may be outside of and proximate to the outer boundary of the flap; and/ or (3) for a cataract removal procedure, marker 50 may outline the lens capsule of the eye.

Also recall that computer 30 determines when the movement of marker 50 is in an alert range and provides a notification in response to the determination. An alert range describes an unacceptable amount of movement of marker 50, which represents an unacceptable amount of movement of eye 22. An unacceptable movement may be movement that misaligns eye 22 with the treatment pattern such that a notification should be provided and/or the procedure should be terminated. An alert range may be expressed as movement of a distance d greater than a maximum acceptable distance Q, expressed in set notation as {d|d>Q}, where Q is any suitable number, e.g., 50 to 500 microns.

In certain embodiments, the alert range comprises a plurality of non-overlapping subset alert ranges that form a partition of the alert range. A partition of a set is collection of disjoint subsets of the set where the union of the subsets equals the set. For example, the alert range {d|d>Q} may be partitioned into {d|Q1<d≤Q2, Q2<d≤Q3, . . . , Qn<d}, where Q=Q1. A subset alert range closer to the maximum acceptable distance Q indicates less misalignment than a subset alert range farther away from the maximum acceptable distance Q. Accordingly, a movement in a subset alert range closer to the maximum acceptable distance Q may be less urgent than a movement in one farther away. In certain embodiments, a subset alert range may be a terminating alert range, which represents unacceptable movement such that the procedure should be terminated.

In certain embodiments, computer 30 determines the alert range corresponding to the surgical procedure. Certain procedures (e.g., creating a lenticule) may require more precision than other procedures (e.g., creating a flap). For example, a lenticule has a shape with refractive properties, so the lenticular incision should be precisely aligned with the treatment pattern. A flap typically has a flat bed incision with no refractive properties, so the alignment need not be as precise. Accordingly, the maximum acceptable distance Q for the alert range may be stricter for a procedure that require more precision. For example, for a lenticule extraction procedure, the maximum acceptable distance Q may be in the range of 50 to 150 microns. As another example, for a flap creation procedure, the maximum acceptable distance Q may be in the range of 100 to 500 microns. As yet another example, for a cataract removal procedure, the maximum acceptable distance Q may be in the range of 50 to 500.

Computer 30 may provide any suitable notification, e.g., audio and/or visual notification. Examples of audio notifications include beeps and spoken words that may be provided via an interface such as an electronic speaker. Examples of visual notifications include lights and graphical icons that may be displayed on an interface such as a display 36 (e.g., a display screen).

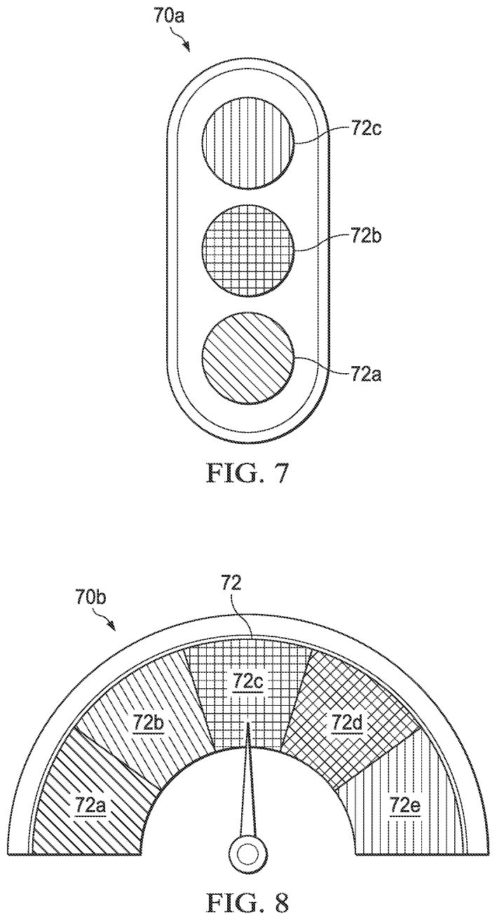

In certain embodiments, computer 30 provides a plurality of notifications for a plurality of subset alert ranges. In some instances, each notification corresponds to a particular subset alert range, and computer 30 provides different notifications for different subset alert ranges, e.g., a first notification for a first subset alert range is distinct from a second notification for a second subset alert range. For example, the first notification may have a first visual characteristic that is distinct from a second visual characteristic of the second notification. As another example, the first notification may have a first audio characteristic that is distinct from a second audio characteristic of the second notification. In certain embodiments, computer 30 terminates the surgical procedure in response to determining that the movement of the marker is in a terminating alert range. Examples of visual notification icons for multiple subset alert ranges are described with reference to FIGS. 7 and 8.

FIG. 7 illustrates an example of a traffic light notification icon 70 (70a) that may be used by system 10 of FIG. 1. A notification icon 70 is a graphical element (which may be presented via display 36) that provides a notification when the movement of marker 50 reaches an alert range. In certain embodiments, notification icon 70 includes alert levels 72 (72a, 72b, 72c). An alert level 72 may correspond to a subset alert range. If the movement of marker 50 falls into a subset alert range, the icon 70 may visually emphasize (e.g., light up, point to, blink, outline, or otherwise emphasize) the corresponding alert level 72.

In the illustrated example, alert level 72a is a green light, alert level 72b is a yellow light, and alert level 72c is a red light. When the movement falls into a subset alert range, icon 70 may emphasize (e.g., light up or make brighter) the corresponding light. Green alert level 72a may correspond to no movement of marker 50 or movement that has not reached an alert level, i.e., the movement is in an acceptable range. Yellow alert level 72b may correspond to the subset alert range closest to the minimum alert value, i.e., the movement is not acceptable, but not to the point that requires terminating the procedure. Red alert level 72c may correspond to a subset alert range that is a terminating alert range, i.e., the movement requires terminating the procedure.

FIG. 8 illustrates an example of a meter notification icon 70 (70b) that may be used by system 10 of FIG. 1. Notification icon 70b includes alert levels 72 (72a to 72e). In the illustrated example, alert level 72a is green; alert level 72b is yellowish green; alert level 72c is yellow; 72d is orange; and alert level 72e is red.

Certain alert levels 72 may correspond to a subset alert range. When the movement of marker 50 falls into a subset alert range, icon 70 may emphasize (e.g., point to) the corresponding level 72. Green alert level 72a may correspond to no movement of marker 50 or movement that has not reached an alert level, i.e., the movement is in the acceptable range. Yellowish green alert level 72b may correspond to the subset alert range closest to the minimum alert value, i.e., the movement is not acceptable, but close to acceptable. Yellow alert level 72c may correspond to the next closest subset alert range, i.e., the movement is not acceptable, but not to the point that requires terminating the procedure. Orange alert level 72d may correspond to the next closest subset alert range, i.e., the movement is not acceptable and close to the point that requires terminating the procedure. Red alert level 72e may correspond to a subset alert range that is a terminating alert range, i.e., the movement requires terminating the procedure.

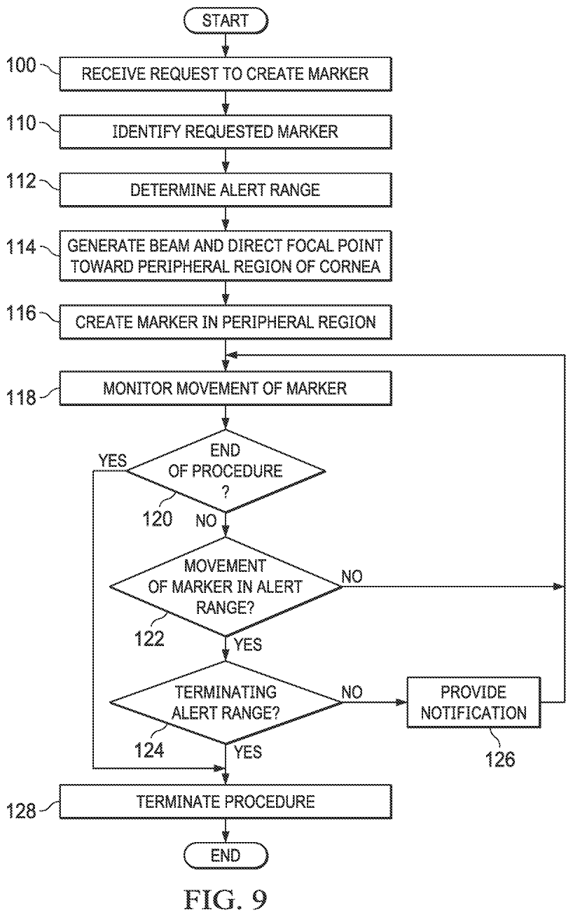

FIG. 9 illustrates an example of a method for creating marker 50 in eye 22 that may be performed by system 10 of FIG. 1. In certain embodiments, computer 30 performs the method by instructing parts of system 10 to perform the actions of the method. Marker 50 may be created during a surgical procedure, e.g., a lenticule extraction, flap creation, or a cataract removal procedure. The method starts at step 100, where computer 30 receives a request to create marker 50 in cornea 46 of eye 40. Marker 50 may have a shape that can indicate translational and/or rotational movement of eye 22. In certain embodiments, the request may be received from a user (e.g., surgeon) of system 10 or may be received from a computer program 34 for an automated surgical procedure.

Computer 30 identifies the requested marker 50 at step 110. In certain embodiments, computer 30 may identify a marker 50 that corresponds to the surgical procedure. For example, a lenticule extraction procedure may use a marker 50 that is external to and proximate to the outer boundary of the lenticule to be extracted. As another example, a flap creation procedure may use a marker 50 that is external to and proximate to the outer boundary of the flap to be created. As yet another example, a cataract procedure may use a marker 50 that outlines the lens capsule of the cataractous lens.

At step 112, computer 30 determines an alert range. In certain embodiments, computer 30 may determine an alert range that corresponds to the surgical procedure. For example, a lenticule extraction procedure may use an alert range with a maximum acceptable distance Q of 50 to 150 microns. As another example, a flap creation procedure may use an alert range with a maximum acceptable distance Q of 100 to 500 microns. As yet another example, a cataract procedure may use an alert range with a maximum acceptable distance Q of 50 to 500 microns.

Computer 30 instructs laser source 12 to generate a laser beam and instructs scanner 16 to direct the focal point of the beam towards peripheral region 48 of cornea 46 at step 114. The controllable components create marker 50 in peripheral region 48 at step 116. Computer 30 monitors the movement of marker 50 at step 118 using camera 38.

The surgical procedure may be completed at step 120. If the procedure has been completed, the method proceeds to step 128, where computer 30 terminates the procedure. If the procedure has not been completed, the method proceeds to step 122.

Computer 30 checks whether the movement of marker 50 is in the alert range at step 122. If the movement is not in the alert range, the method returns to step 118, where computer 30 continues to monitor the movement of marker 50. If the movement is in the alert range, the method proceeds to step 124.

At step 124, computer 30 checks whether the alert range is a terminating alert range. A terminating alert range indicates when the surgical procedure should be terminated. For example, the terminating range may indicate the eye is so misaligned that the treatment will not be effective. If the alert range is not a terminating alert range, the method proceeds to step 126.

At step 126, computer 30 provides a notification that the movement is in the alert range. In certain embodiments, the alert range has multiple non-overlapping subset alert ranges that form a partition of the alert range, and computer 30 provides a notification for each subset alert range. In certain cases, computer 30 provides distinct notifications for different subset ranges. For example, a first notification for a first subset alert range may be distinct from a second notification for a second subset alert range. The notifications may be visually distinct (e.g., the first notification has a first visual characteristic that is distinct from a second visual characteristic of the second notification) or aurally distinct (e.g., the first notification has a first audio characteristic that is distinct from a second audio characteristic of the second notification). The method then returns to step 118, where computer 30 continues to monitor the movement of marker 50.

If the alert range is a terminating alert range at step 124, the method proceeds to step 128, where computer 30 terminates the procedure. The method then ends.

A component (e.g., control computer 30) of the systems and apparatuses disclosed herein may include an interface, logic, and/or memory, any of which may include computer hardware and/or software. An interface (e.g., display 36) can receive input to the component and/or send output from the component, and is typically used to exchange information between, e.g., software, hardware, peripheral devices, users, and combinations of these. A user interface (e.g., a Graphical User Interface (GUI)) is a type of interface that a user can utilize to interact with a computer. Examples of interfaces include a display screen, touchscreen, keyboard, mouse, gesture sensor, microphone, and speakers.

Logic (e.g., logic 31) can perform operations of the component. Logic may include one or more electronic devices that process data, e.g., execute instructions to generate output from input. Examples of such an electronic device include a computer, processor, microprocessor (e.g., a Central Processing Unit (CPU)), and computer chip. Logic may include computer software that encodes instructions capable of being executed by the electronic device to perform operations. Examples of computer software include a computer program, application, and operating system.

A memory (e.g., memory 32) can store information and may comprise tangible, computer-readable, and/or computer-executable storage medium. Examples of memory include computer memory (e.g., Random Access Memory (RAM) or Read Only Memory (ROM)), mass storage media (e.g., a hard disk), removable storage media (e.g., a Compact Disk (CD) or Digital Video or Versatile Disk (DVD)), database, network storage (e.g., a server), and/or other computer-readable media. Particular embodiments may be directed to memory encoded with computer software.

Although this disclosure has been described in terms of certain embodiments, modifications (such as changes, substitutions, additions, omissions, and/or other modifications) of the embodiments will be apparent to those skilled in the art. Accordingly, modifications may be made to the embodiments without departing from the scope of the invention. For example, modifications may be made to the systems and apparatuses disclosed herein. The components of the systems and apparatuses may be integrated or separated, or the operations of the systems and apparatuses may be performed by more, fewer, or other components, as apparent to those skilled in the art. As another example, modifications may be made to the methods disclosed herein. The methods may include more, fewer, or other steps, and the steps may be performed in any suitable order, as apparent to those skilled in the art.

To aid the Patent Office and readers in interpreting the claims, Applicants note that they do not intend any of the claims or claim elements to invoke 35 U.S.C. § 112(f), unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the applicants to refer to structures known to those skilled in the relevant art and is not intended to invoke 35 U.S.C. § 112(f).

What is claimed:

1. An ophthalmic surgical system for creating a marker in a cornea of an eye for a surgical procedure, comprising:
a plurality of controllable components comprising:
a laser source configured to generate a laser beam having a plurality of ultrashort pulses;
a scanner configured to transversely and longitudinally direct a focal point of the laser beam; and
an objective configured to focus the focal point through a patient interface towards the eye;
a camera configured to image movement of the eye; and
a computer configured to:
create the marker in the cornea, the marker having a shape that can indicate rotational movement of the eye, by:
instructing the scanner to transversely and longitudinally direct the focal point towards a peripheral region of the cornea;
instructing one or more of the controllable components to create the marker in the peripheral region of the cornea;
determine that movement of the marker is in an alert range, the alert range indicating an unacceptable amount of movement of the marker; and
provide one or more notifications in response to determining that the movement of the marker is in the alert range.

2. The ophthalmic surgical system of claim 1, wherein the shape of the marker is selected from one or more of the following: a polygon, a line, a plurality of lines, a plurality of lines intersecting at a point, a plurality of lines intersecting at a plurality of points, a circle with a line, an oval, and one or more alphanumeric characters.

3. The ophthalmic surgical system of claim 1, the computer configured to:
identify the marker corresponding to the surgical procedure; and
create the identified marker in the cornea.

4. The ophthalmic surgical system of claim 3, wherein:
the surgical procedure is a lenticule extraction procedure; and
the computer is configured to:
identify that the marker is external to and proximate to an outer boundary of a lenticule to be extracted; and
create the marker external to and proximate to the outer boundary.

5. The ophthalmic surgical system of claim 3, wherein:
the surgical procedure is a flap creation procedure; and
the computer is configured to:
identify that the marker is external to and proximate to an outer boundary of a flap to be created; and
create the marker external to and proximate to the outer boundary.

6. The ophthalmic surgical system of claim 3, wherein:
the surgical procedure is a cataract removal procedure; and
the computer is configured to:
identify that the marker outlines a lens capsule of the eye; and
create the marker that outlines the lens capsule of the eye.

7. The ophthalmic surgical system of claim 1, wherein a notification of the one or more notifications is an audio notification.

8. The ophthalmic surgical system of claim 1, wherein a notification of the one or more notifications is a visual notification.

9. The ophthalmic surgical system of claim 1, wherein:
the alert range comprises a plurality of non-overlapping subset alert ranges that form a partition of the alert range; and
the computer is configured to provide a plurality of notifications, each notification corresponding to a subset alert range, a first notification for a first subset alert range distinct from a second notification for a second subset alert range.

10. The ophthalmic surgical system of claim 9, wherein the first notification has a first visual characteristic that is distinct from a second visual characteristic of the second notification.

11. The ophthalmic surgical system of claim 9, wherein the first notification has a first audio characteristic that is distinct from a second audio characteristic of the second notification.

12. The ophthalmic surgical system of claim 9, wherein:
a subset alert range of the plurality of subset alert ranges is a terminating alert range; and
the computer is configured to terminate the surgical procedure in response to determining that the movement of the marker is in the terminating alert range.

13. The ophthalmic surgical system of claim 9, wherein:
the ophthalmic surgical system further comprises a display screen; and
the computer is configured to:
display a notification icon comprising a plurality of alert levels on the display screen, at least one alert level corresponding to a subset alert range; and
visually emphasize the at least one alert level in response to determining that the movement of the marker is in the corresponding subset alert range.

14. The ophthalmic surgical system of claim 1, the computer further configured to determine the alert range corresponding to the surgical procedure.

15. The ophthalmic surgical system of claim 14, wherein:
the surgical procedure is a lenticule extraction procedure; and
the alert range has a maximum acceptable distance of 50 to 150 microns.

16. The ophthalmic surgical system of claim 14, wherein:
the surgical procedure is a flap creation procedure; and
the alert range has a maximum acceptable distance of 100 to 500 microns.

17. The ophthalmic surgical system of claim 14, wherein:
the surgical procedure is a cataract removal procedure; and
the alert range has a maximum acceptable distance of 100 to 500 microns.

18. A method for creating a marker in a cornea of an eye for a surgical procedure, comprising:
creating, by a computer, the marker in the cornea, the marker having a shape that can indicate rotational movement of the eye, by:
instructing a scanner to transversely and longitudinally direct a focal point of a laser beam towards a peripheral region of the cornea, the laser beam having a plurality of ultrashort pulses; and
instructing one or more of a set of controllable components to create the marker in the peripheral region of the cornea, the set of controllable components comprising a laser source configured to generate the laser beam, the scanner configured to transversely and longitudinally direct the focal point of the laser beam, and an objective configured to focus the focal point through a patient interface towards the eye;
determining, by the computer, that movement of the marker is in an alert range, the alert range indicating an unacceptable amount of movement of the marker; and
providing, by the computer, one or more notifications in response to determining that the movement of the marker is in the alert range.

19. The method of claim 18, further comprising:
identifying the marker corresponding to the surgical procedure; and
creating the identified marker in the cornea.

20. The method of claim 18:
wherein the alert range comprises a plurality of non-overlapping subset alert ranges that form a partition of the alert range; and
further comprising:
providing a plurality of notifications, each notification corresponding to a subset alert range, a first notification for a first subset alert range distinct from a second notification for a second subset alert range;
displaying a notification icon comprising a plurality of alert levels on a display screen, at least one alert level corresponding to a subset alert range; and
visually emphasizing the at least one alert level in response to determining that the movement of the marker is in the corresponding subset alert range.
